(19) United States
(12) Patent Application Publication
(10) Pub. No.: US 2025/0241718 A1
(43) Pub. Date: Jul. 31, 2025

(54) ACTIVELY FACILITATED INSTRUMENT RETRACTION

(71) Applicant: Intuitive Surgical Operations, Inc., Sunnyvale, CA (US)

(72) Inventors: Shibing Liu, San Jose, CA (US); Samuel B. Schorr, San Jose, CA (US)

(73) Assignee: Intuitive Surgical Operations, Inc., Sunnyvale, CA (US)

(21) Appl. No.: 19/027,709

(22) Filed: Jan. 17, 2025

Related U.S. Application Data
(60) Provisional application No. 63/622,261, filed on Jan. 18, 2024.

(51) Int. Cl. A61B 34/30; A61B 90/00

(52) U.S. Cl. CPC A61B 34/30; A61B 90/06; A61B 2034/301; A61B 2034/303; A61B 2090/067

(57) ABSTRACT

A medical system includes a manipulator assembly that includes a first and second actuator. The first actuator drives an articulable body portion of a flexible elongate device in a first direction along an articulation degree of freedom by increasing tension on a first pull wire of the articulable body portion. The second actuator drives the articulable body portion in a second direction along the articulation degree of freedom opposite the first direction by increasing tension on a second pull wire of the articulable body portion. The medical system further includes a control system that causes the articulable body portion to enter a bent state by increasing tension on the first pull wire, detects a retraction of the flexible elongate device while the flexible elongate device is in the bent state, and based on the detection of the retraction, counters the bent state by increasing tension on the second pull wire.

### CROSS-REFERENCE TO RELATED APPLICATIONS

This application claims priority to and the benefit of the filing date of U.S. Provisional Patent Application 63/622,261, filed on Jan. 18, 2024, which is hereby incorporated by reference herein in its entirety.

### FIELD

Disclosed embodiments relate to improved robotic and/or medical devices, systems, and methods.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter or bronchoscope, that can be inserted into anatomic passageways and navigated toward a region of interest within the patient anatomy.

The flexible elongated device may be articulable, facilitating navigation of the flexible elongate device within the patient anatomy. After the articulation, the flexible elongate device may not necessarily return to a neutral position. In absence of a return to the neutral position, a geometric mismatch between the flexible elongate device in its bent state and the surrounding anatomical structure is likely to occur, e.g., when retracting the flexible elongate device, causing contact between the flexible elongate device and the surrounding anatomical structure during the retraction of the flexible elongate device. This may result in significant contact forces.

### SUMMARY

The following presents a simplified summary of various examples described herein and is not intended to identify key or critical elements or to delineate the scope of the claims.

In some examples, a medical system comprises a manipulator assembly comprising: a first actuator configured to drive an articulable body portion of a flexible elongate device in a first direction along an articulation degree of freedom by increasing tension on a first pull wire of the articulable body portion; and a second actuator configured to drive the articulable body portion in a second direction along the articulation degree of freedom opposite the first direction by increasing tension on a second pull wire of the articulable body portion; and a control system coupled to the manipulator assembly, the control system configured to: cause the articulable body portion to enter a bent state based on controlling the first actuator to increase tension on the first pull wire, detect a retraction of the flexible elongate device while the flexible elongate device is in the bent state, and based on the detection of the retraction, counter the bent state by controlling the second actuator to increase tension on the second pull wire.

In some examples, a non-transitory machine-readable medium comprises a plurality of machine-readable instructions executed by one or more processors associated with a medical system, the medical system comprising: a manipulator assembly comprising: a first actuator configured to drive an articulable body portion of a flexible elongate device in a first direction along an articulation degree of freedom by increasing tension on a first pull wire of the articulable body portion, and a second actuator configured to drive the articulable body portion in a second direction along the articulation degree of freedom opposite the first direction by increasing tension on a second pull wire of the articulable body portion, and wherein the plurality of machine-readable instructions causes the one or more processors to perform a method comprising: causing the articulable body portion to enter a bent state based on controlling the first actuator to increase tension on the first pull wire; detecting a retraction of the flexible elongate device while the flexible elongate device is in the bent state; and based on the detection of the retraction, countering the bent state by controlling the second actuator to increase tension on the second pull wire.

In some examples, a method for operating a medical system comprises a manipulator assembly comprising: a first actuator configured to drive an articulable body portion of a flexible elongate device in a first direction along an articulation degree of freedom by increasing tension on a first pull wire of the articulable body portion, and a second actuator configured to drive the articulable body portion in a second direction along the articulation degree of freedom opposite the first direction by increasing tension on a second pull wire of the articulable body portion, and the method comprising: causing the articulable body portion to enter a bent state based on controlling the first actuator to increase tension on the first pull wire; detecting a retraction of the flexible elongate device while the flexible elongate device is in the bent state; and based on the detection of the retraction, countering the bent state by controlling the second actuator to increase tension on the second pull wire.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within a flexible elongate device according to some embodiments.

FIGS. 3A and 3B are simplified diagrams of side views of a patient coordinate space including a medical instrument mounted on an insertion assembly according to some embodiments.

FIG. 4 is a flowchart of a method according to some embodiments.

FIGS. 5A, 5B, and 5C are flowcharts of a method according to some embodiments.

FIGS. 6A and 6B are simplified diagrams of control loops according to some embodiments.

FIGS. 7A and 7B are illustrations of an actively facilitated instrument retraction according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Embodiments of the disclosure include medical systems and methods for operating such medical systems. Medical systems may be medical systems that use flexible elongate devices (e.g., catheters, bronchoscopes, endoscopes, etc.), but also other medical systems.

A medical system may include a medical instrument, and the medical instrument may be driven along one or more degrees of freedom. In some embodiments, the driving may involve an actuator driven movement of the medical instrument along an insertion degree of freedom. Driving along the insertion degree of freedom may involve insertion and/or retraction of the flexible elongate device, e.g., inside an anatomical structure such as a lung. In some embodiments, the driving may involve an actuator driven movement of the medical instrument along one or more articulation degrees of freedom. Driving along the articulation degrees of freedom may involve articulation of an articulable body portion of the flexible elongate device, such as a distal portion of the flexible elongate device. In one example, the articulation degrees of freedom include articulation across pitch and yaw axes. Driving along the articulation degrees of freedom may be performed to navigate the distal end of the flexible elongate device toward a target tissue and/or when orienting an end effector towards target tissue to perform a medical operation such as a biopsy, an ablation, an electroporation, etc. The articulation of the articulable body portion may be driven from a proximal portion of the elongate device, e.g., using pull wires, driven by actuators. The pull wires may be actively put under mechanical tension by the actuators to cause the articulation.

Embodiments of the disclosure may use an antagonistic actuation scheme. For example, two pull wires, on opposite sides, may control the articulation along a single axis. In one embodiment, there are 4 pull wires total, with pairs of pull wires controlling orthogonal pitch and yaw axes. The pull wires may all exert a “minimum tension” that is set to ensure responsiveness of the controls but not be too high so as to potentially damage or quickly degrade the pull wires.

To articulate along an axis, one of the pull wires of the pair (call “active pull wire”) is brought to a higher tension, while the other pull wire (call “passive pull wire”) is kept at the minimum tension. This results in the articulable portion being bent. To relax, the active pull wire that was brought to the higher tension is brought to a lower tension, such as the minimum tension (which may be a pre-specified non-zero tension or a zero tension), while the passive pull wire is kept at the minimum tension. This may be sufficient to cause the articulable body portion to straighten, although not necessarily, for example, in presence of internal friction or low bending stiffness. Essentially, the bending stiffness of the flexible elongate device may provide a spring-like force that facilitates a return towards the neutral (non-articulated) position. However, with at least some amount of internal friction associated with articulating the elongate device being inevitable, the articulable body portion of the flexible elongate device may remain in its bent state and may not return to the neutral position (or sufficiently neutral position) when the actuators are controlled to release the tension on the pull wires. For a flexible elongate device that has a lower bending stiffness (i.e., less spring-like force), the remaining in the bent state may be particularly pronounced with no return to the neutral position occurring at all. For a flexible elongate device that has a higher bending stiffness (i.e., more spring-like force), an incomplete/partial return towards the neutral position may occur.

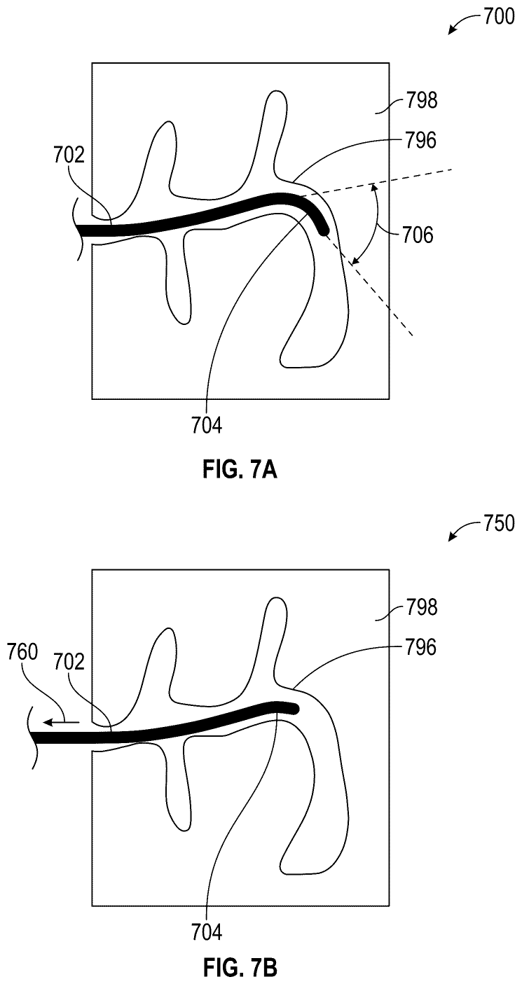

In absence of a return to the neutral position, a geometric mismatch between the flexible elongate device in its bent state and the surrounding anatomical structure is likely to occur (e.g., as illustrated in FIG. 7A), causing contact between the articulable body portion of the flexible elongate device and the surrounding anatomical structure during the retraction. This may result in significant contact forces.

Embodiments of the disclosure actively facilitate a return from the bent state of the flexible elongate device towards the neutral position, thereby reducing contact forces between the flexible elongate device at the articulable body portion and the surrounding anatomical structure during retraction (e.g., as illustrated in FIGS. 7A and 7B). The facilitating of the return may involve an active return from the bent state with an active straightening of the articulable body portion. The active straightening is not limited to relaxing the tension on the active pull wire (e.g., to minimum tension), but, in addition, increases the tension on the passive pull wire (e.g., to be higher than minimum tension). Both pull wires may be relaxed (e.g., to the minimum tension), when the articulable body portion is sufficiently straightened,

Once the articulable body portion is sufficiently straightened (e.g., as illustrated in FIG. 7B), the risk of a collision with the surrounding anatomical structure is reduced, and even if a collision occurs, the resulting contact forces may be reduced, compared to contact forces resulting from a retraction in the bent state. Alternatively, the facilitating of the return from the bent state may involve a partial compensation of the internal friction of the flexible elongate device. Although the partial compensation of the internal friction does not immediately result in a return from the bent state to a neutral position, it does reduce the contact forces that are needed for a backdriving of the articulable body portion towards the neutral position, when the articulable body portion comes in contact with the surrounding anatomical structure.

A more detailed discussion is provided below in reference to the figures.

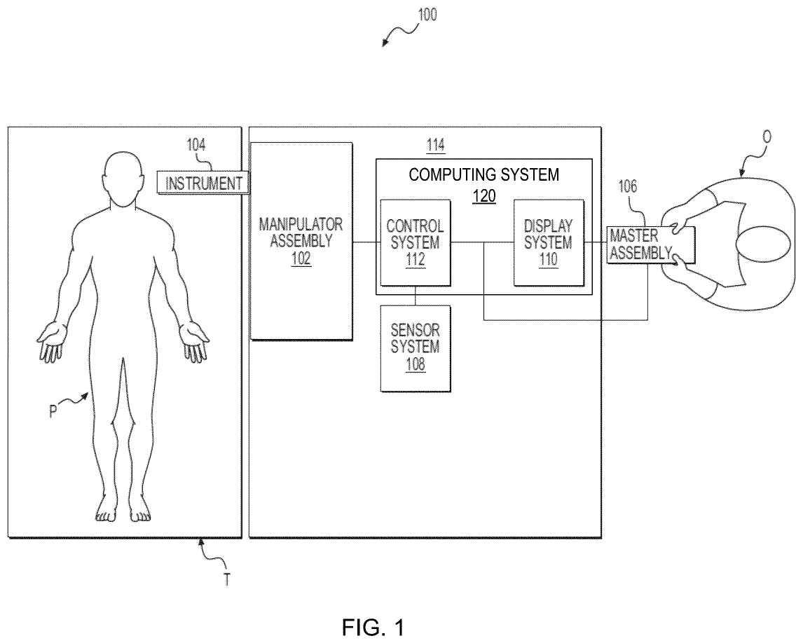

Turning to the figures, FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the medical instrument 104 may be controlled directly by the operator O. In some examples, the manipulator assembly 102 may be manually controlled by the operator O. Direct operator control may include various handles and operator interfaces for hand-held operation of the medical instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016, and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

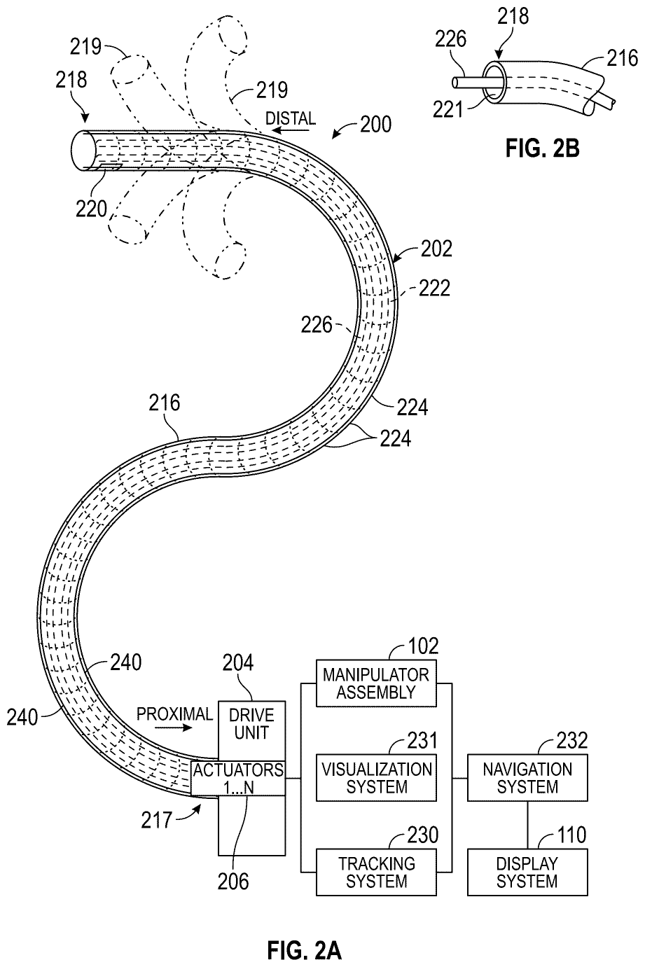

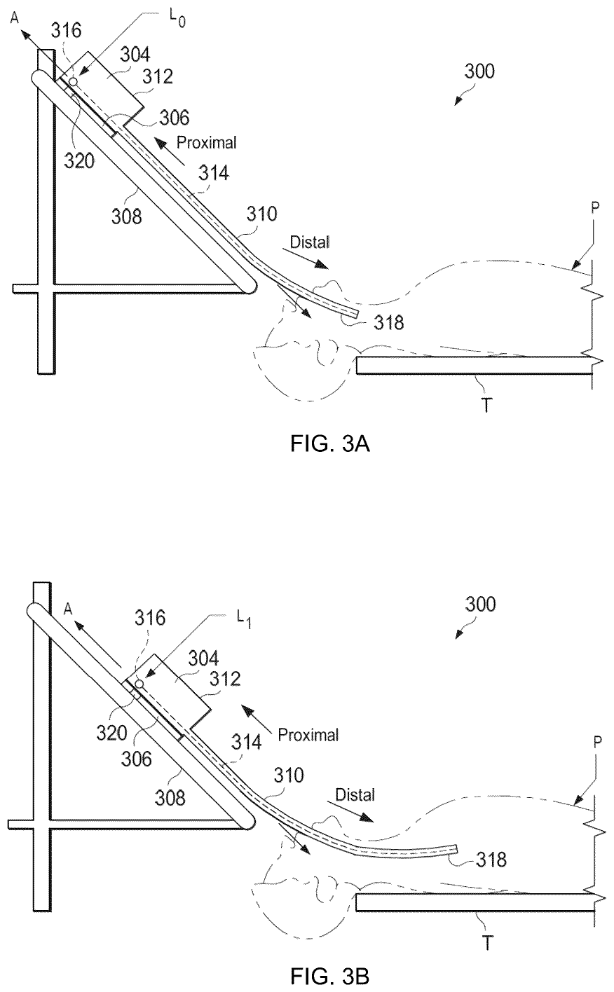

FIG. 2A is a simplified diagram of a medical instrument system 200 according to some embodiments. The medical instrument system 200 includes a flexible elongate device 202 (also referred to as elongate device 202), a drive unit 204, and a flexible tool, e.g., a medical tool, 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, tool recognition sensor 233, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel or lumen 221 through which a flexible tool, e.g., the medical tool, 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. The distal end 218 may, thus, form the articulable body portion. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019, and titled “Flexible Elongate Device Systems and Methods”), which is incorporated by reference herein in its entirety.

When configured to operate as antagonists, pairs of actuators 206 (e.g., one pair of actuators for pitch control of the distal portion 218 and one pair of actuators for yaw control of the distal portion 218), may be used to articulate the distal portion 218 and control a stiffness of the flexible body 216. Further, by maintaining a minimum level of tension on the pull-wires 240, slack in the pull-wires 240 may be avoided. Releasing or reducing the force on the pull-wires 240 of the flexible catheter 202 may cause a corresponding reduced stiffness or rigidity in the flexible catheter 202. Similarly, applying or increasing a pulling force in the pull-wires 240 of the flexible body 216 may cause an increase in stiffness or rigidity of the flexible catheter 202. For example, the flexible body 216 may become stiffer with multiple steering pull-wires being pulled concurrently. The stiffness or rigidity of the flexible catheter 202 may be a closed-loop stiffness or rigidity controlled by the control system. Examples of a closed-loop catheter control system and methods are described, for example, in U.S. patent application Ser. No. 13/274,198 (filed Oct. 14, 2011) (disclosing “Catheters with Control Modes for Interchangeable Probes”) which is incorporated by reference herein in its entirety.

Medical instrument system 200 may include the tracking system for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexible body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999, and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor system 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the flexible tool 226 within the elongate device 202 according to some embodiments. The flexible body 216 of the elongate device 202 may include the lumen 221 sized and shaped to receive the flexible tool 226. In some embodiments, the flexible tool 226 may be used for procedures such as diagnostics, imaging, surgery, biopsy, ablation, illumination, irrigation, suction, electroporation, etc. Flexible tool 226 can be deployed through channel or lumen 221 of flexible body 216 and operated at a procedural site within the anatomy. Flexible tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the flexible tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The flexible tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the lumen 221 when the biopsy tool is within the lumen 221. The flexible tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of flexible tool 226 to facilitate simultaneous image capture and tissue intervention, such as within the same lumen 221 or in separate channels. A flexible tool 226 may be advanced from the opening of the lumen 221 to perform the procedure (or some other functionality) and then retracted back into the lumen 221 when the procedure is complete. The flexible tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 215 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005, and titled “Articulated Surgical Instrument for Performing Minimally Invasive Surgery with Enhanced Dexterity and Sensitivity”) and U.S. Pat. No. 9,259,274 (filed Sep. 30, 2008, and titled “Passive Preload and Capstan Drive for Surgical Instruments”), which are incorporated by reference herein in their entireties.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. The drive unit 204 may further include brakes. One brake may be paired with one actuator. In configurations that pair an actuator with a gear reducer, the brake may be located on the actuator side, which enables even a relatively small brake to produce a significant braking force. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety.

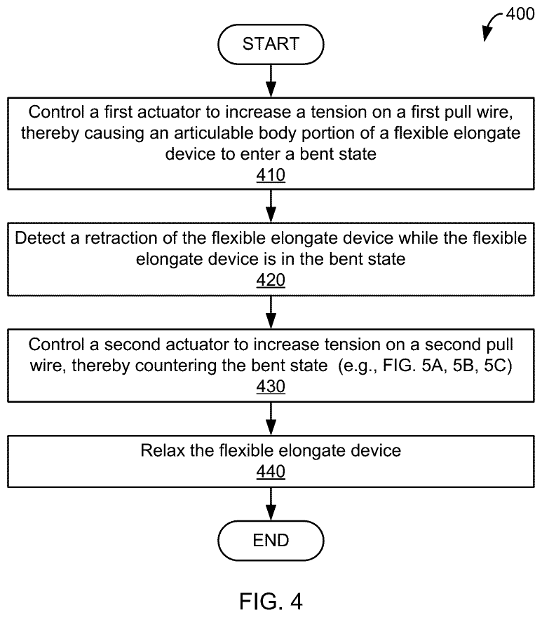

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308. The instrument carriage 306 or insertion stage 308 may further include brakes. One brake may be paired with one actuator. For example, an actuator may be provided for driving the medical instrument along the insertion axis of the manipulator assembly, and a brake may be provided for inhibiting movement of the medical instrument along the insertion axis.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

Embodiments of the disclosure actively facilitate a retraction of the flexible elongate device, e.g., in a transition from the configuration as shown in FIG. 3B to the configuration shown in FIG. 3A. Methods as subsequently discussed may be used.

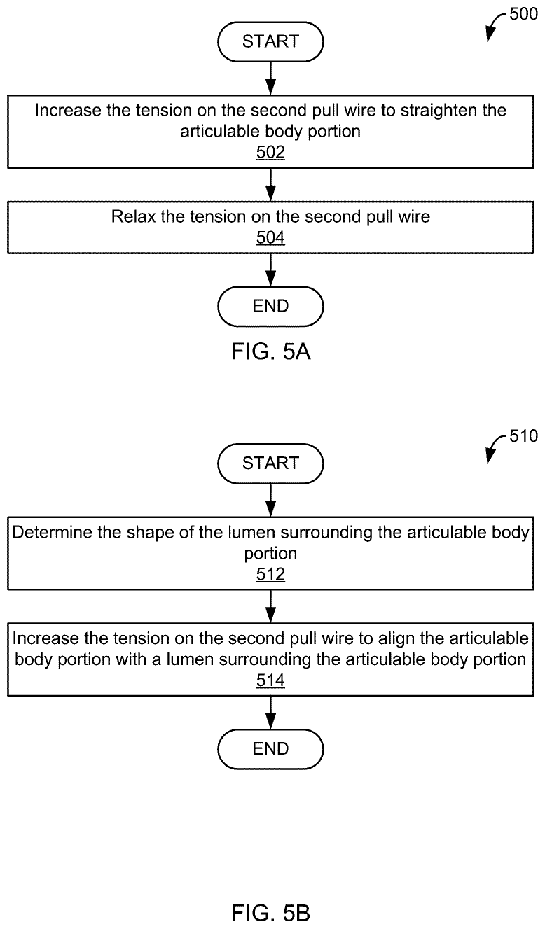

FIG. 4 shows a flowchart of a method 400 for facilitating retraction of a flexible elongate device, in accordance with embodiments of the disclosure. The method may be used during retraction of the flexible elongate device, thereby reducing the potential of significant contact forces between the flexible elongate device and a mismatching geometry of the surrounding environment (e.g., tissue), as illustrated in FIGS. 7A and 7B.

The method may be implemented using instructions stored on a non-transitory medium that may be executed by a computing system, e.g., the computing system 120.

While the various blocks in FIG. 4 are presented and described sequentially, some or all of the blocks may be executed in different orders, may be combined or omitted, and some or all of the blocks may be executed in parallel. Furthermore, the blocks may be performed actively or passively.

For the purpose of discussing the flowchart, it is assumed that the flexible elongate device has been advanced into the patient as shown in FIG. 3B, e.g., to perform a diagnostic or therapeutic procedure. Further, for the sake of simplicity, the described operations are for an articulation along a single articulation degree of freedom, controlled by two antagonistically operating actuators. A first actuator may cause articulation of the articulable body portion in a first direction of the degree of freedom, and a second actuator may cause articulation of the articulable body portion in a second direction of the degree of freedom, opposite to the first direction. Embodiments of the disclosure are equally applicable to configurations with any number of articulation degrees of freedom, e.g., for a configuration that includes pitch and yaw articulation degrees of freedom. An articulation along a diagonal axis may be achieved by simultaneous, coordinated articulations along both the pitch and yaw articulation degrees of freedom. Depending on the amount of articulation along pitch and yaw degrees of freedom, an articulation in any direction may be achieved. Further, articulations along the pitch and yaw degrees of freedom may be performed independently, e.g., at different times.

In block 410, the articulable body portion of the flexible elongate device is caused to enter a bent state. The bent state may be reached by controlling the first actuator to increase tension on a first pull wire of the articulable body portion. At the same time, the second actuator, antagonistically operating with the first actuator, may be controlled to keep the tension on the second pull wire at a minimum tension when the tension on the first pull wire is increased to enter the bent state. In another example, the second actuator may be controlled to reduce tension in a second pull wire of the articulable body portion to a minimum tension, thereby facilitating the entering of the bent state.

The articulation may be in response to a user command (e.g., provided using a control device) to articulate the articulable body portion. The articulation may be for various reasons, e.g., to orient the articulable body portion towards a target site, to orient the articulable body portion along a navigation path within surrounding lumen), thereby allowing further insertion of the flexible elongate device, etc. An example is shown in FIG. 7A.

In block 420, a retraction of the flexible elongate device is detected. Any method for detecting the retraction may be used without departing from the disclosure. For example, the retraction may be detected based on a user input of a retraction command (e.g., provided using a control device). Alternatively, the retraction may be detected using a sensor (e.g., a position sensor system configured to sense an insertion depth of the flexible elongate device). The flexible elongate device may be in the bent state when the retraction is detected.

In block 430, based on the detection of the retraction, the bent state of the articulable body portion is countered by controlling the second actuator to increase tension on the second pull wire of the articulable body portion. Controlling the second actuator to increase tension on the second pull wire, in comparison to merely relaxing the pull wires, in some embodiments, actively facilitates a return of the articulable body portion to a neutral (or less bent) state. While the relaxation of the pull wire(s) alone, in some cases may not be sufficient to counter the bent state, the increase in tension on the second pull wire may enables a countering of the bent state, even in presence of internal friction or low bending stiffness of the flexible elongate device. The tension on the second pull wire may be increased from the tension on the second pull wire in block 410 when tension on the first pull wire was increased to cause the articulable body portion to enter the bent state. For example, the tension on the second pull wire may be increased from the minimum tension. The countering of the bent state may further involve controlling the first actuator to decrease the tension on the first pull wire to facilitate the countering of the bent state. The relaxing of the tension in the first pull wire may be synchronized with the increasing of the tension in the second pull wire. The tension in the first pull wire may be reduced to no lower than the minimum tension.

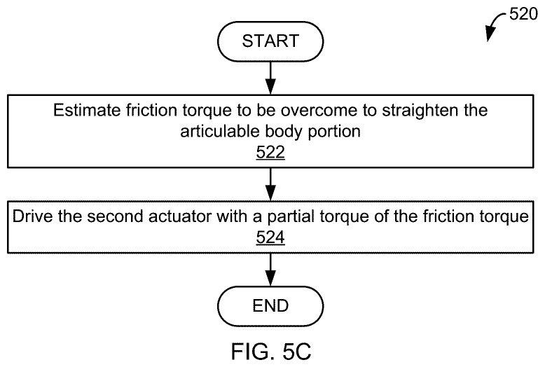

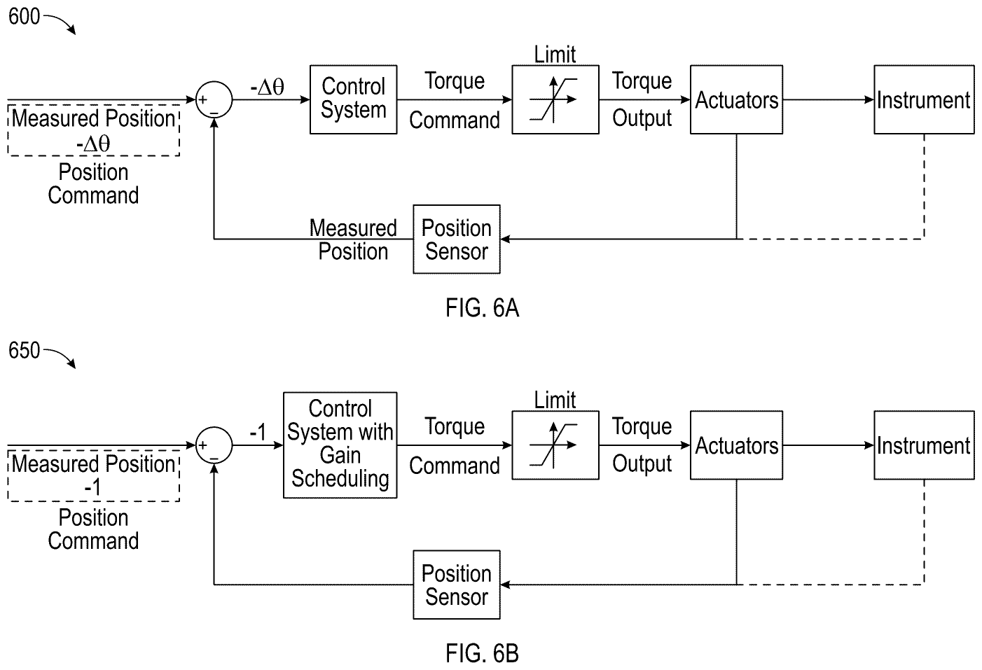

In some embodiments, the countering of the bent state is performed to reduce the magnitude of contact forces that may be present between the flexible elongate device and a mismatching geometry of the surrounding environment, in case of mechanical contact. An example that illustrates the countering of the bent state based on a detection of a retraction is shown in FIG. 7B. The countering of the bent state may be performed in different manners, as discussed below in reference to the flowcharts of FIGS. 5A, 5B, and 5C. Example control architectures that may be used to counter the bent state are shown in FIGS. 6A and 6B.

In block 440, optionally, after countering the bent state, the flexible elongate device may be relaxed by placing both the first and the second pull wires at the minimum tension or zero tension. In combination, when executed during a retraction of the flexible elongate device, the execution of blocks 430 and 440 result in an active facilitating of the straightening of the flexible elongate device, followed by a relaxing of the flexible elongate device. When relaxed, the flexible elongate device may passively alter its shape during the retraction, driven by external forces resulting from the flexible elongate device coming in contact with a surrounding lumen. A detailed description of the operations that may be performed in block 440 is provided below in reference to FIG. 5A.

Execution of the method 400, in accordance with embodiments of the disclosure, addresses issues that may otherwise result from retraction of the flexible elongate catheter with the articulable body portion in the bent state. For example, the execution of the method 400 may avoid significant contact forces between the articulable body portion and the surrounding environment during retraction. These issues may be addressed even for flexible elongate devices that have significant internal friction and/or low bending stiffness (i.e., flexible elongate devices that are particularly unlikely to return to a neutral or straight configuration on their own after release of the tension on the pull wires), based on operations 430 and 440 that actively facilitate the return of the flexible elongate device to a neutral configuration. Additional details are subsequently described in reference to the flowcharts of FIGS. 5A, 5B, and 5C which capture different approaches to countering the bent state of the articulable body portion.

In some embodiments, the manner in which the method 400 is executed may depend on various factors. Accordingly, the method 400 may involve conditional execution of certain operations. Specifically, for example, as previously noted, the countering of the bent state in block 430 may be performed as described in FIGS. 5A, 5B, and 5C. Criteria used for selecting the operations to be performed in block 430 may include the shape of the flexible elongate device and the inserted length of the flexible elongate device. For example, a high degree of curvature or tortuosity of a deeply inserted flexible elongate device may result in significant friction forces that would be encountered when attempting to passively straighten the articulable body portion. This suggests the use of an active straightening of the articulable body portion (FIG. 5A) or a continued alignment of the articulable body portion with the surrounding lumen (FIG. 5B). In contrast, for a less deeply inserted flexible elongate device and/or less tortuosity, a partial friction compensation (FIG. 5C) may be sufficient. Furthermore, for scenarios that involve even less friction (limited insertion depth and/or minimal tortuosity), none of the operations of FIGS. 5A, 5B, and 5C may be executed, resulting in a purely passive retraction of the flexible elongate device. In some embodiments, a decision logic may be used to select between the described methods, e.g., based on thresholds for insertion length, tortuosity, and/or other factors. A detailed discussion of frictional effects is provided below in reference to FIG. 5C.

FIG. 5A shows a flowchart of a method 500 for controlling the second actuator to increase the tension on the second pull wire to counter the bent state of the articulable body portion.

In block 502, the tension on the second pull wire is increased to straighten the articulable body portion. Straightening the articulable body portion may involve a straightening by any amount ranging from a slight straightening to a complete straightening. In one example, the straightening is performed until the articulable body portion is near the neutral position, e.g., within 10°. In some embodiments, a commanded torque suitable to cause the straightening of articulable body portion is determined. The second actuator may then be controlled using the commanded torque. The determining of the commanded torque may be performed as described below in reference to FIGS. 6A and 6B.

In block 504, the tension on the second pull wire, after straightening of the articulable body portion, may be relaxed during the retraction of the flexible elongated device. In one embodiment, the flexible elongate device, once the tensions on the first and second pull wires are relaxed (e.g., to the minimum tension), may be in a passive mode in which external forces, e.g., caused by contact with a surrounding lumen, may passively alter the orientation of the articulable body portion for the remainder of the retraction through the lumen.

FIG. 5B shows a flowchart of a method 510 for controlling the second actuator to increase the tension on the second pull wire to counter the bent state of the articulable body portion.

In operation 512, the shape of the lumen surrounding the articulable body portion is determined. The shape of the lumen may be determined based on a model (e.g., a 3D model) of the patient anatomy derived from pre-operative or intra-operative images obtained as previously described. Thus, based on the tracked location of the flexible elongate device being registered (e.g., dynamically referenced) with the model, the shape of the lumen at the articulable body portion may be determined, even while the flexible elongate device is inserted or retracted. Additionally or alternatively, the shape of the lumen may be estimated based on a navigation history (e.g., the history of articulation commands used to insert the flexible elongate device, based on the assumption that during the insertion, articulation was generally controlled to follow the shape of the lumen).

In block 514, the tension on the second pull wire is increased to align the articulable body portion with the lumen surrounding the articulable body portion. Accordingly, while both the method 500 and 510 have in common that they actively decrease the bending angle of the articulable body portion, method 510 performs an adjustment of the articulable body portion in an environment-dependent manner, whereas method 500 straightens the articulable body portion regardless of the surrounding environment. For example, using the method 510, if the articulable body portion is surrounded by a curved section of a lumen, the articulable body portion may be aligned to match the curvature, e.g., of an airway within a lung.

Depending on the shape of the lumen at the articulable body portion, it may be necessary to increase rather than decrease the articulation of the articulable body portion. The increased articulation may be obtained by controlling the first actuator to increase tension on the first pull wire of the articulable body portion. At the same time, the second actuator, antagonistically operating with the first actuator, may be controlled to keep the tension on the second pull wire at a minimum or reduced tension when the tension on the first pull wire is increased to increase articulation.

Bocks 512 and 514 may be continuously performed during the retraction of the flexible elongate device, thereby causing the articulable body portion to follow the shape of the lumen during the ongoing retraction. In other words, the articulable body portion may be actively and continuously steered. The steering may involve control of both the first and the second actuator as needed for the articulable body portion to follow the lumen, during retraction. The steering requires knowledge of the shape of the lumen at the current position of the articulable body portion as the flexible elongate device is being retracted, in order to enable generation of the appropriate commands to the first and second actuators. Such knowledge may be available from a registration with an anatomical model, as previously described.

In some embodiments, a commanded torque suitable to cause the straightening or steering of articulable body portion is determined. The second actuator or both the first and second actuators (for steering in both directions of the articulation) may then be controlled using the commanded torque. The determining of the commanded torque may be performed as described below in reference to FIGS. 6A and 6B.

FIG. 5C shows a flowchart of a method 520 for controlling the second actuator to increase the tension on the second pull wire to counter the bent state of the articulable body portion. Unlike the methods 500, 510, which may rely on a feedback loop, method 520 uses an open loop or feedforward approach.

In operation 522, a friction torque that the second actuator would need to overcome to straighten the articulable body portion in the articulation degree of freedom is estimated. As illustrated in FIG. 2A, a pull wire between an actuator and the articulable body portion is routed through the flexible elongate device, where the pull wire may be, at least partially, in contact with element of the flexible elongate device, such as elements of a pull wire guide system. While a completely straight-running pull wire in the flexible elongate device may experience relatively little friction, a bend in the flexible elongate device may introduce non-negligible additional friction as a result of the pull wire having to be guided in presence of the bend. The bend, particularly when the pull wire is under tension, results in contact forces between the pull wire and other elements of the flexible elongate device, at the bend. The friction may increase with sharper bends, with multiple (e.g., alternating) bends, an increased bending angle, etc. In some embodiments, the friction torque is correlated with a total or cumulative bending angle (tortuosity) of the flexible elongate device. The effect of the total bending angle and potentially other factors such as the tension on the pullwire may be represented by models, lookup tables, etc., which may have been obtained either empirically or through simulation. Generally speaking, an increase in the total bending angle results in an increase in friction, although the model or lookup table may establish a considerably more complex relationship. Other factors may include, for example, the stiffness of the flexible elongate device. A stiffer flexible elongate device with its tendency to return to its originally neutral shape may produce a stiffness torque that reduces the torque that the second actuator needs to supply to cause a straightening of the articulable body portion. More generally, in some embodiments, a relationship between the shape of the flexible elongate device and the friction torque is established, thereby enabling an estimation of the friction torque based on a total bending angle of the flexible elongate device.

The shape of the flexible elongate device may be obtained in different manners. For example, an output of the previously described shape sensor may be used. Alternatively, based on a known insertion depth and an anatomy model, the shape may be determined. The insertion depth may be obtained using, for example, the previously discussed position sensor system and/or a known or sensed position of the instrument carriage, whereas the anatomy model may have been or may be obtained using different image modalities such as a computed tomography, a fluoroscopy, etc.

In operation 524, the second actuator is driven with a partial torque of the friction torque estimated in operation 522, e.g., by commanding a current corresponding to the partial torque. For example, the second actuator may apply 70-80% of the friction torque. In other words, operation 524 performs a partial friction compensation. Assuming that the estimate of the friction torque is accurate, a straightening of the articulable body portion does not occur, because the actual friction torque is higher than the torque applied by the second actuator. However, with the actual friction torque being partially compensated for, a relatively small external force at the articulable body portion may be sufficient to cause a straightening of the articulable body portion. Accordingly, a collision of the articulable body portion with the surrounding environment during the retraction may result in significantly reduced forces that would cause a straightening of the articulable body portion. The method 520 is, thus, distinct form the methods 500 and 510 because it counters the bent state to an extent that is limited to not necessarily causing a straightening of the articulable body portion, unless an external force that acts on the articulable body portion facilitates the straightening. In contrast, the methods 500 and 510 may involve some degree of straightening (ranging from a minimal straightening to a complete straightening) of the articulable body portion in absence of such an external force.

In an alternative embodiment, the entire friction torque is used to drive the second actuator. In this case, movement of the articulable body portion may occur, even in absence of an external force. The friction torque may be limited within a range, to avoid undesirable excessive movement of the articulable body portion in case of an inaccurate estimate of the friction torque.

Operations 522 and 524 may be continuously performed during the retraction of the flexible elongate device, thereby continuously countering the bent state of the articulable body portion as the articulable body portion follows the shape of the lumen during the ongoing retraction. In other words, a partial friction compensation may be actively and continuously performed. The friction compensation may involve control of both the first and the second actuator as needed for the articulable body portion to follow the lumen, during retraction.

Furthermore, in some embodiments, operations 522 and/or 524 are conditionally executed. For example, a test may be performed to determine whether the current degree of articulation of the articulable body portion (e.g., expressed as a bending angle) exceeds a first threshold value. If the first threshold value is not exceeded, operation 522 and 524 may be skipped because the articulable body portion is not sufficiently bent to benefit from any straightening. However, if the current degree of articulation exceeds the first threshold value, another test may be performed to determine whether the current total bending angle exceeds a second threshold value. If the second threshold value is not exceeded, operation 522 and 524 may be skipped because the total bending angle is not sufficiently high to cause a friction torque that would result in undesirable contact forces when the articulable body portion comes into contact with the surrounding environment. In this case, an acceptable contact force would cause a straightening of the articulable body portion. However, if the current total bending angle exceeds the second threshold value, operations 522 and 524 may be performed to avoid elevated contact forces during the retraction.

FIGS. 6A and 6B are simplified diagrams of control loops 600, 650, according to some embodiments. In the examples as shown, the control loops are position control loops. The control loops may be used to drive the actuators of the medical system. For example, the control loops may be used to drive the second actuator in operation 502 of method 500, and in operation 512 of method 510, but also to drive the first actuator. Both control loops are designed to cause an actuator to generate a commanded torque by driving the position control loop with a positional offset, as subsequently discussed. The control loops 600, 650 are alternative implementations for controlling the actuators.

Both control loops, 600, 650, may be understood as position control loops in which a difference between a position command and a measured position is used to generate a position error that is fed into a controller (e.g., a PD or PID controller), which generates a torque command. The torque command, after an amplitude limiting may be used to command a motor current to the actuator. In some embodiments, the measured position is obtained using a position sensor. The position sensor may sense at the motor (angle of the motor shaft) or at the articulable body portion (degree of articulation). In some embodiments a control loop as shown may exist for each actuator.

Referring to FIG. 6A, the control loop 600 receives a modified position command. In some embodiments, the modified position command is the measured position, modified by a specified angle difference −Δθ. Accordingly, the input to the controller is the increment −Δθ, i.e., a positional offset (or orientational offset). The specified angle difference may specify a small angle, e.g., 3°, to ensure that the articulable body portion straightens smoothly and gradually. Alternatively, the input to the controller may be the difference between position command and measured position, which results in a more brisk adjustment of the articulable body portion. In either case, the torque command generated by the controller may be sufficient to overcome the friction torque, thus resulting in movement of the articulable body portion.

In some embodiments, the angle difference may be modulated based on an estimate of the friction torque. The estimate of the friction torque may be obtained as previously described. A higher friction torque may result in an up-adjustment of the angle difference, whereas a lower friction torque may result in a down-adjustment of the angle difference, in order to obtain the desired adjustment of the articulable body portion. The magnitude of the angle difference to be applied may further depend on the proportional control gain, Kₚ, of the controller. A smaller angle difference may be sufficient for a higher Kₚ, whereas a larger angle difference may be necessary for a lower Kₚ.

Referring to FIG. 6B, the control loop 650 as shown is a modified version of the control loop 600 shown in FIG. 6A. In some embodiments, the control loop 650 receives a modified position command. In some embodiments, the modified position command is the measured position, modified by a constant, e.g., −1. Accordingly, the input to the controller is the constant. In other words, in the example of FIG. 6B, the control loop 650 is biased with a constant positional offset.

In some embodiments, the controller is a controller with gain scheduling, for example, of the proportional control gain, Kₚ. The modulation of the gain scheduling may be based on the specified angle difference as discussed in reference to FIG. 6A. Accordingly, the control loops 600 and 650 may be considered equivalent.

In some embodiments, the position control loop includes a damping. The damping may limit a rate of position change of the articulable body portion. More specifically, the controllers of the control loops 600, 650 may optionally include a non-zero damping, e.g., in the form of a damping term K_{d}, to smoothen the straightening movement of the articulable body portion.

FIGS. 7A and 7B are illustrations of a facilitated instrument retraction 700, 750, according to some embodiments. In both FIGS. 7A and 7B, a flexible elongate device 702 is driven within a lumen 796 of a surrounding anatomical structure 798. The elongate flexible device 702 may be driven using a configuration as previously described in reference to FIGS. 3A and 3B. As previously described, the flexible elongate device has an articulable body portion 704. In FIG. 7A, the flexible elongate device 702 is inserted to an insertion depth that necessitates an articulation of the articulable body portion 704 to a bent state 706. A retraction 760 is illustrated in FIG. 7B. As a result of the retraction, the articulable body portion has been straightened to at least some degree from the bent state 706. The straightening may have occurred using the methods and control loops described in reference to FIGS. 4, 5A, 5B, 5C, 6A, and 6B.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM), a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed is:

1. A medical system comprising:
a manipulator assembly comprising:
a first actuator configured to drive an articulable body portion of a flexible elongate device in a first direction along an articulation degree of freedom by increasing tension on a first pull wire of the articulable body portion; and
a second actuator configured to drive the articulable body portion in a second direction along the articulation degree of freedom opposite the first direction by increasing tension on a second pull wire of the articulable body portion; and
a control system coupled to the manipulator assembly, the control system configured to:
cause the articulable body portion to enter a bent state based on controlling the first actuator to increase tension on the first pull wire,
detect a retraction of the flexible elongate device while the flexible elongate device is in the bent state, and
based on the detection of the retraction, counter the bent state by controlling the second actuator to increase tension on the second pull wire.

2. The medical system of claim 1, wherein controlling the second actuator to increase tension on the second pull wire comprises increasing the tension on the second pull wire to straighten the articulable body portion in the articulation degree of freedom.

3. The medical system of claim 2, wherein the control system is further configured to, after increasing the tension on the second pull wire to straighten the articulable body portion in the articulation degree of freedom, controlling the second actuator to relax the tension on the second pull wire during the retraction.

4. The medical system of claim 2, wherein the control system is further configured to determine a commanded torque, required to straighten the articulable body portion by the second actuator.

5. The medical system of claim 4, wherein the commanded torque is based on a friction torque to be overcome when straightening the articulable body portion in the articulation degree of freedom.

6. The medical system of claim 5, wherein the control system is further configured to estimate the friction torque based on a total bending angle of the flexible elongate device.

7. The medical system of claim 5, wherein the control system is further configured to cause the second actuator to generate the commanded torque by driving a position control loop of the second actuator with a positional offset.

8. The medical system of claim 7, wherein the position control loop comprises a damping term that limits a rate of position change.

9. The medical system of claim 5, wherein the control system is further configured to cause the second actuator to generate the commanded torque by biasing a position control loop of the second actuator with a constant positional offset and modulating a control gain of the position control loop.

10. The medical system of claim 1, wherein controlling the second actuator to increase tension on the second pull wire comprises increasing the tension on the second pull wire to align the articulable body portion in the articulation degree of freedom with a lumen surrounding the articulable body portion.

11. The medical system of claim 1, wherein the control system is further configured to estimate a friction torque to be overcome to straighten the articulable body portion in the articulation degree of freedom, and
wherein controlling the second actuator to increase tension on the second pull wire comprises driving the second actuator to apply a partial torque of the friction torque.

12. The medical system of claim 11, wherein the control system is further configured to estimate the friction torque based on a total bending angle of the flexible elongate device.

13. The medical system of claim 12, wherein the control system is further configured to:
detect that the total bending angle is below a preset threshold, and
based on the detection, stop driving the second actuator.

14. The medical system of claim 1, wherein the control system is further configured to:
when increasing the tension on the second pull wire to counter the bent state, control the first actuator to decrease tension on the first pull wire.

15. The medical system of claim 14, wherein the tension on the first pull wire is decreased to a minimum tension.

16. The medical system of claim 1, wherein the control system is further configured to:
when increasing the tension on the first pull wire to enter the bent state, control the second actuator to keep the tension on the second pull wire at a minimum tension.

17. The medical system of claim 16, wherein increasing the tension on the second pull wire to counter the bent state comprises increasing the tension on the second pull wire from the minimum tension.

18. A non-transitory machine-readable medium comprising a plurality of machine-readable instructions executed by one or more processors associated with a medical system, the medical system comprising:
a manipulator assembly comprising:
a first actuator configured to drive an articulable body portion of a flexible elongate device in a first direction along an articulation degree of freedom by increasing tension on a first pull wire of the articulable body portion, and
a second actuator configured to drive the articulable body portion in a second direction along the articulation degree of freedom opposite the first direction by increasing tension on a second pull wire of the articulable body portion, and
wherein the plurality of machine-readable instructions causes the one or more processors to perform a method comprising:
causing the articulable body portion to enter a bent state based on controlling the first actuator to increase tension on the first pull wire;
detecting a retraction of the flexible elongate device while the flexible elongate device is in the bent state; and
based on the detection of the retraction, countering the bent state by controlling the second actuator to increase tension on the second pull wire.

19. The non-transitory machine-readable medium of claim 18, wherein controlling the second actuator to increase tension on the second pull wire comprises increasing the tension on the second pull wire to straighten the articulable body portion in the articulation degree of freedom.

20. A method for operating a medical system, comprising:
a manipulator assembly comprising:
a first actuator configured to drive an articulable body portion of a flexible elongate device in a first direction along an articulation degree of freedom by increasing tension on a first pull wire of the articulable body portion, and
a second actuator configured to drive the articulable body portion in a second direction along the articulation degree of freedom opposite the first direction by increasing tension on a second pull wire of the articulable body portion, and
the method comprising:
causing the articulable body portion to enter a bent state based on controlling the first actuator to increase tension on the first pull wire;
detecting a retraction of the flexible elongate device while the flexible elongate device is in the bent state; and
based on the detection of the retraction, countering the bent state by controlling the second actuator to increase tension on the second pull wire.
